(19) United States
(12) Patent Application Publication
(10) Pub. No.: US 2017/0119519 A1
(43) Pub. Date: May 4, 2017

(54) DEVICE WITH A J-SHAPED END AND WITH AN INFLATABLE BALLOON FOR USE AS SUPPORT FOR DEVICES FOR TISSUE RECONSTRUCTION OF EXCISED TISSUES OF THE RENAL PELVIS AND/OR OF THE URETEROPELVIC JUNCTION

(71) Applicants: Antonio SAMBUSSETI, Cremona (IT); Gianni CANCARINI, Brescia (IT)

(72) Inventors: Antonio SAMBUSSETI, Cremona (IT); Gianni CANCARINI, Brescia (IT)

(21) Appl. No.: 15/317,636

(22) PCT Filed: Jun. 8, 2015
(86) PCT No.: PCT/EP2015/062734

§ 371 Date: Dec. 9, 2016

(30) Foreign Application Priority Data
Jun. 12, 2014 (IT) MI2014A 001070

(51) Int. Cl. A61F 2/04; A61M 27/00; A61F 2/00

(52) U.S. Cl. CPC A61F 2/04; A61F 2/0063; A61M 27/008; A61F 2002/048; A61F 2210/0004; A61F 2230/0067

(57) ABSTRACT

A support device (10) includes a flexible cannula (1), substantially cylindrical and hollow, which extends from a curled proximal end (3) to a distal end (2) provided with an expandable balloon (5), for use as support of a conical device (100) constructed during a surgical operation from a patch in resorbable fabric (1), for use in the replacement and reconstruction of excised tissues of a renal pelvis and/or of a ureteropelvic junction so as to facilitate the tissue reconstruction thereof in the original form.

The present invention relates to a device with an end of the J type and provided with an inflatable balloon for use as support of devices for use in the replacement and reconstruction of tissues removed of the renal pelvis and/or of the ureteropelvic junction.

More particularly the present invention relates to such a device, similar to a stent one, to be used as support of a patch in resorbable fabric for use in the replacement and reconstruction of tissues of the renal pelvis and/or of the ureteropelvic junction in the case of stenosis, neoplasms or other types of diseases that obstruct the flow of urine.

The upper part of the ureter, that in direct contact with the kidney, takes the name of renal pelvis and represents the anatomical entity which holds the urine produced by the kidney, conveying it into the ureter.

The renal pelvis has a shape roughly comparable to a funnel crushed in anteroposterior direction whose base turned upwards and outwards is divided into various recesses (calyces) which are held in the kidney while its narrowed end or apex is turned downwards, continuing into the ureter. The point of connection between said renal pelvis and said ureter is defined as ureteropelvic junction.

It happens at times that said pelvis and/or said ureteropelvic junction are affected by stenosis, neoplasms or other types of diseases which obstruct the flow of urine, therefore requiring a surgical operation.

For example the ureteropelvic junction has very often a narrowing which determines an obstacle to the normal flow of urine which goes to accumulate in the renal pelvis, causing the dilation thereof. This dilation causes a compression of the kidney with damage which can even lead to the functional loss of the same.

It is therefore necessary to provide, in this situation, a surgical operation of pyeloplasty, in order to remodel the ureteropelvic junction by removal of the stenotic segment and subsequent reconstructive plastic surgery so as to re-create a passage for the urine of adequate calibre.

This remodelling, which takes place by removing part of the tissue and performing successive sutures, to give thereto the shape and the dimension required, may be unsuitable in the case wherein the autologous tissues to be used for the reconstruction are weakened or thin for congenital or pathological reasons.

Much more rarely it may occur that the renal pelvis is the site of serious neoplasms, such as urothelial carcinoma: in this case the necessary surgical removal of the portion of renal pelvis affected by the pathology entails a disadvantageous reduction in the volume of the pelvis.

It is therefore highly desirable in the field of pathologies of the renal pelvis and/or of the ureteropelvic junction to have available a biocompatible device which can replace the stenotic tissue removed and which also allows the growth of the autologous tissue.

US 2012/316656 describes a stent for treating the obstructions of a ureter comprising an elongated element which has a distal end, a proximal end and a medial portion placed between the two ones, wherein the medial portion is provided with an expandable element configured to be inserted in the ureter of the patient and configured to be in contact with the ureter to contribute to maintaining the elongated element in position inside the patient.

WO97/16218 describes a stent for treating the obstructions of a ureter and/or ureteropelvic junction comprising a system for treating the ureter and/or the ureteropelvic junction, comprising: means for widening an obstruction, means for guiding the widening means through the urethra, bladder and ureter, wherein the guide means can be introduced into the patient through the urethra, bladder and ureter to extend beyond the ureteropelvic junction.

The object of the present invention is that of overcoming, at least in part, the disadvantages of the prior art by providing a device for the replacement and regrowth of autologous tissues of renal pelvises and/or ureteropelvic junctions following stenosis, neoplasms or other types of diseases which obstruct the flow of urine.

These and other objects are achieved by the device in accordance with the invention having the features listed in the appended independent claim 1.

Advantageous embodiments of the invention are disclosed by the dependent claims.

An object of the present invention relates to a support device with a curled end and the other end provided with an inflatable balloon, for use as support in the tissue reconstruction and replacement of the renal pelvis and/or of the ureteropelvic junction, in association with a patch in resorbable fabric so as to facilitate the reconstruction, in the original form, of the removed tissues.

Said device, including the balloon, is in flexible plastic material, for example polyurethane, silicone or another biocompatible material, and can also be in these materials but coated completely with pyrolytic turbostratic carbon so as to have a greater resistance to contact with urine.

When the balloon is deflated, the device can be inserted along the ureter as far as the pelvis like a typical double-j stent remaining in position thanks to the curled tip which prevents the device from being displaced, once inserted in the bladder (similarly to an “autostatic” stent). When the balloon is inflated, the device can conform to the removed part of the renal pelvis and/or of the ureteropelvic junction acting as support for a patch in bioabsorbable fabric, for example PGA, to be wound around this balloon and to be sutured to the edges of the tissue zone of the renal pelvis and/or of the ureteropelvic junction which has been removed.

In practice the abovementioned device has found to be suitable for acting as scaffold or frame for a patch in resorbable fabric when the latter is covered by growing autologous cells generated by the process of tissue reconstruction of the patient, once the patch has been implanted in the patient.

After having inflated the balloon, the present stent device can remain in place for a brief period (days or weeks) or for a longer period of time (weeks or months) according to the extent of the zone removed.

Further features of the invention will be made clearer by the following detailed description, referred to a purely non-limiting example thereof, illustrated in the accompanying drawings in which:

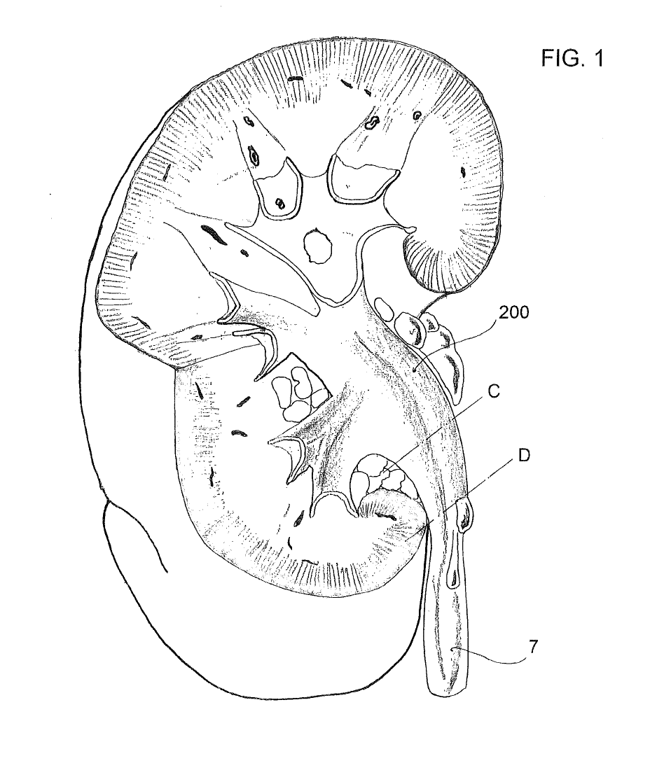

FIG. 1 is a partially sectioned front view of a kidney;

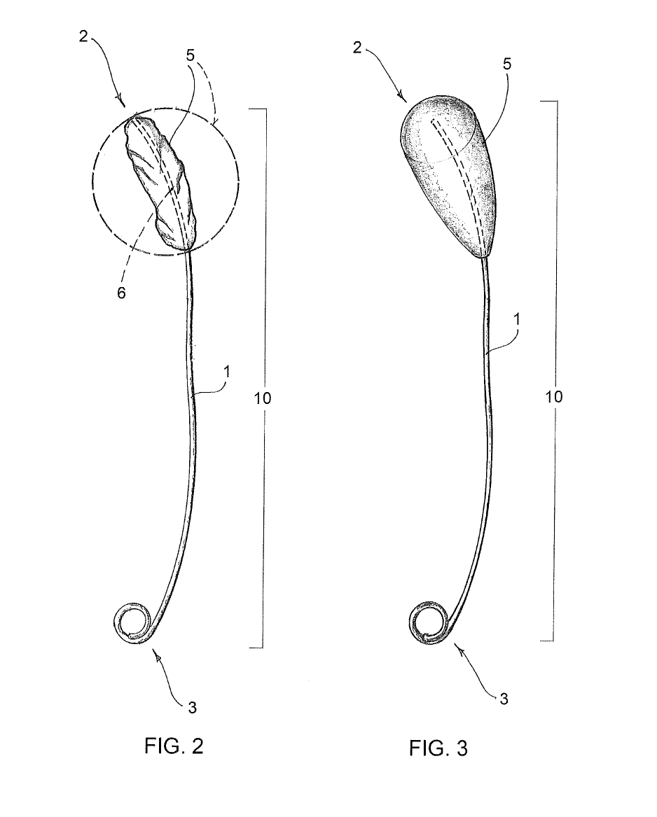

FIG. 2 is a front view of the stent-like device in accordance with the invention in the configuration of deflated balloon, where the inflated balloon of round shape has been shown as dotted;

FIG. 3 is a front view of the stent-like device in accordance with the invention where the inflated balloon has a conical shape;

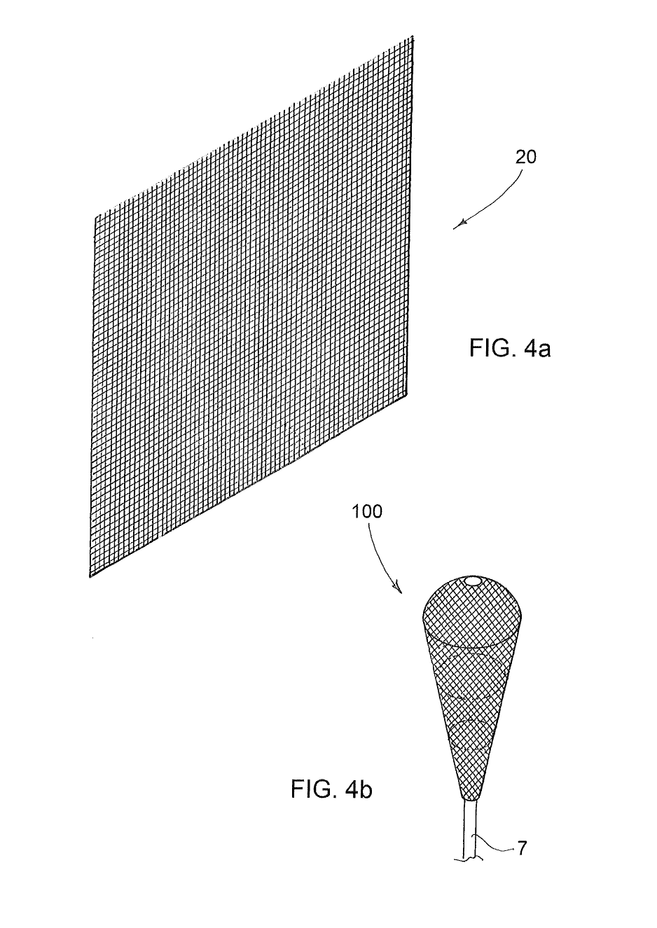

FIGS. 4a and 4b are perspective views of the fabric and of the corresponding scaffold device to be used in association with the stent-like device in accordance with the invention.

With the aid of FIGS. 1-4 a description is given of a support stent-like device for the replacement and reconstruction of tissues of the renal pelvis and/or of the ureteropelvic junction in accordance with the present invention, denoted overall by reference numeral 10.

The stent support device 10 comprises a cannula 1, substantially cylindrical and hollow, which extends from a proximal end 3 with a curled tip to a distal end 2.

The curled tip 3 is apt to act as stop against the entrance of the ureter 7, similarly to the double-j type stents, while the distal end 2 is intended to be inserted in the ureteropelvic junction and in the renal pelvis.

Said cannula 1 is constituted by two coaxial channels (not illustrated), arranged along its entire length: a first channel, more internal, is opened at the two ends so as to allow the urine to flow out along the ureter 7 towards the bladder, once the device 10 has been inserted in the ureter 7.

The second channel, more external, is connected to a balloon, or cap, expandable, 5, provided at the distal end 2 of the device 10 at its end section.

Said balloon 5 is suitable for being inflated with sterile bidistilled water or with sterile physiological solution, once placed inside the pelvis and/or the junction as will be described in detail here below.

This cap 5 and its ends are sealed on the cannula 1 according to known techniques used in the sector of medical devices, creating in this way an expandable chamber around said end section 2 of the cannula 1.

The cap 5 is made in flexible and plastic material suitable for forming films, such as for example polyethylene (PE), with low or high density, polypropylene (PP), PVC free from phthalates (DEHP-free PVC), polyurethane (PU), silicone.

Said cap 5 can also be subsequently coated with a microfilm of pyrolytic turbostratic carbon with thickness of approximately 0.2-0.3 microns.

It is understood that the aforesaid materials can be used also to construct the cannula 1 of the device 10, just as the coating with pyrolytic turbostratic carbon can be carried out also on the cannula 1.

Said expandable cap 5 is in fluid communication with the more external channel of said cannula through one or more holes 6 placed only in the section of the more external channel which is surrounded by said cap 5.

Via said through holes 6 it is therefore possible to expand partially or completely the closed chamber after having injected a fluid, for example a physiological solution, inside the more external channel of the cannula 1.

It should be noted that the length of the more external channel section surrounded by said cap 5 is not binding for the purpose of the present invention.

The increase in the diameter of said expandable chamber 5 finds a limit in the maximum diameter of expansion of said chamber which is defined by the manufacturer to occupy the zone of the pelvis without widening it.

The diameter of maximum expansion, or inflation, of said expandable cap 5 is such as to create a chamber with a capacity of about 70-100 cc.

The abovementioned measurements are only indicative and not limiting for the purpose of the present invention.

The more internal channel also has holes provided along its entire length with the exception of the section where the balloon 5 is mounted: said holes have the purpose of facilitating the flow of urine from the pelvis 200 towards the bladder via the ureter 7.

The present device generally has a length of about 26-30 cm (for adults) with a diameter of about 4-10 Ch, similarly to ureter catheters of the double-j or pigtail type.

A description will now be given of the procedure of use of the present device 10 in an operation.

First of all a surgical operation is carried out to remove the area of the pelvis 200 (FIG. 1) affected by the stenosis, for example by cutting it in two distinct points along the same number of cutting planes C and D (FIG. 1).

After which, during the operation, the device 10 with deflated balloon is inserted in the bladder and along the entire ureter 7: once the section covered by the balloon 5 has reached the ureteropelvic junction or a zone of the pelvis 200 and the curled end 3 has stopped against the initial lumen of the ureter 7, the expandable cap 5 is inflated up to the diameter of expansion required.

Once the chamber 5 of the device 10 has been inflated, the removed stenotic zone can be reconstructed according to the following technique: a patch of resorbable fabric 20 (FIG. 4a), for example in PGA, is wound around the balloon 5 so as to form a structure with similar shape to the removed zone, for example a truncated cone 100 (FIG. 4b), then suturing the upper and lower edges of said truncated cone 100 to the edges of the tissue zone removed.

In this case the balloon 5 acts as support structure or frame for the truncated cone 100 formed by the resorbable fabric 20, which is to be maintained in the position wherein it has been sutured until the reconstruction of the new tissue. In this way said truncated cone 100 is allowed to maintain this shape even under the weight of the growing new tissue of the renal pelvis and/or of the ureteropelvic junction above said truncated cone 100.

Once the tissue of the pelvis has been reconstructed, generally in around 1 month, the present device will be extracted.

For the sutures a suture yarn in resorbable material (PGA) is preferably used, with dimensions of approximately 4/0, in order to have times of resorption similar to the patch in PGA fabric.

The holes of passage of the suture stitches in the natural tissue do not constitute a risk of leaks of liquid, in that in a few hours the tissue is reconstructed. To avoid leaks of urine (liquid), the holes of the suture stitches are in any case sealed and closed with one cc (a drop) of surgical glue, such as for example Glubran 2TM, normally available commercially.

As patch in PGA fabric, the one described by the Applicant in the patent application MI2009A002093, incorporated here in full for reference, can be used for example.

The present invention is not limited to the particular embodiments previously described and illustrated in the accompanying drawings, but numerous detailed changes may be made thereto, within the reach of the person skilled in the art, without thereby departing from the scope of the invention itself as defined in the appended claims.

1-10. (canceled)

11. Implant for use in the tissue reconstruction and replacement of excised tissues of a renal pelvis and/or of a ureteropelvic junction so as to facilitate the reconstruction thereof in the original natural form, said implant comprising
(A) a support device (10) provided with a shaped end of j-type comprising flexible cannula (1) substantially cylindrical and hollow, which extends from a curled proximal end (3) having a (j) shape to a distal end (2) provided with an expandable balloon (5)
(B) a resorbable conical device (100) constructed during an operation from a patch in resorbable fabric (20) and wound around said balloon (5) of said support device (10),
said resorbable conical device (100) being for use in the tissue reconstruction and replacement of excised tissues of a renal pelvis and/or of a ureteropelvic junction so as to facilitate the reconstruction thereof in the original natural form,
wherein said cannula (1) of said support (10) is constituted by two coaxial channels arranged along its entire length,
a first, more internal channel, being open at the two ends for the flow of urine from the kidney to the bladder;
a second, more external channel, being in fluid communication with said expandable balloon (5).

12. Implant according to claim 11, wherein said support device (10) is made in a flexible plastic material.

13. Implant according to claim 12, wherein the plastic material of said support—device (10) is selected from low or high density polyethylene (PE), polypropylene (PP), PVC, phthalates-free PVC (DEHP free PVC), polyurethane (PU), silicone.

14. Implant according to claim 11, wherein said expandable balloon (5) of said support device (10) is in fluid communication with the more external channel of said cannula (1) through one or more holes (6) arranged on said more external channel only at the section thereof surrounded by said balloon (5) .

15. Implant according to claim 11, wherein the diameter of maximum expansion, or inflation, of said balloon (5) of said support device (10) is such as to create a chamber with a capacity of about 70-100 cc.

16. Implant according to claim 11, wherein the length of said support device (10) is about 26-30 cm (for adults) with a diameter of about 4-10 Ch.

17. Implant according to claim 12, wherein the flexible plastic material of said support device (10) is coated completely with pyrolytic turbostratic carbon.

18. Implant according to claim 13, wherein the plastic material of said support device (10) is polyurethane or silicone.

19. Implant according to claim 13, wherein the plastic material of said support device (10) is coated with a microfilm of pyrolytic turbostratic carbon with a thickness of approximately 0.2-0.3 microns.

20. Device (10) provided with a j-shaped end for supporting a resorbable conical device (100) for use in the tissue reconstruction and replacement of excised tissues of a renal pelvis (200) and/or of a ureteropelvic junction so as to facilitate the reconstruction thereof in the original natural form,
said support device (10) comprising
a flexible cannula (1), substantially cylindrical and hollow, which extends from a curled proximal end (3) having said (j) shape to a distal end (2) provided with an expandable balloon (5), said cannula (1) being constituted by two channels arranged along its entire length,
the first, more internal channel, being open at the two ends for the flow of urine from the kidney to the bladder,
the second, more external channel, being in fluid communication with said expandable balloon (5) through one or more holes (6) arranged on said more external channel only at the section thereof surrounded by said balloon (5),
wherein
the two channels of said cannula (1) are coaxial, and
the first, more internal channel, has also holes along its entire length with the exception of the section where the balloon (5) is mounted in order to facilitate the flow of urine from said pelvis (200) towards the bladder via ureter.

21. Implant according to claim 12, wherein said expandable balloon (5) of said support device (10) is in fluid communication with the more external channel of said cannula (1) through one or more holes (6) arranged on said more external channel only at the section thereof surrounded by said balloon (5).

22. Implant according to claim 13, wherein said expandable balloon (5) of said support device (10) is in fluid communication with the more external channel of said cannula (1) through one or more holes (6) arranged on said more external channel only at the section thereof surrounded by said balloon (5).

23. Implant according to claim 12, wherein the diameter of maximum expansion, or inflation, of said balloon (5) of said support device (10) is such as to create a chamber with a capacity of about 70-100 cc.

24. Implant according to claim 13, wherein the diameter of maximum expansion, or inflation, of said balloon (5) of said support device (10) is such as to create a chamber with a capacity of about 70-100 cc.

25. Implant according to claim 14, wherein the diameter of maximum expansion, or inflation, of said balloon (5) of said support device (10) is such as to create a chamber with a capacity of about 70-100 cc.

26. Implant according to claim 12, wherein the length of said support device (10) is about 26-30 cm (for adults) with a diameter of about 4-10 Ch.

27. Implant according to claim 13, wherein the length of said support device (10) is about 26-30 cm (for adults) with a diameter of about 4-10 Ch.

28. Implant according to claim 14, wherein the length of said support device (10) is about 26-30 cm (for adults) with a diameter of about 4-10 Ch.

29. Implant according to claim 15, wherein the length of said support device (10) is about 26-30 cm (for adults) with a diameter of about 4-10 Ch.

30. Implant according to claim 17, wherein the plastic material of said support device (10) is polyurethane or silicone.
